(19) United States
(12) Patent Application Publication
(10) Pub. No.: US 2020/0069926 A1
(43) Pub. Date: Mar. 5, 2020

(54) METHOD AND APPARATUS FOR EMBEDDING AN IMPLANT IN A BALLOON SURFACE BY INDUCTIVE HEATING OF THE IMPLANT

(71) Applicant: BIOTRONIK AG, Buelach (CH)

(72) Inventor: AMIR FARGAHI, BUELACH (CH)

(21) Appl. No.: 16/537,682

(22) Filed: Aug. 12, 2019

(30) Foreign Application Priority Data
Aug. 28, 2018 (DE) 102018120940.1

(51) Int. Cl. A61M 25/10; A61F 2/958; B29C 65/32; B29C 65/00; B29C 33/38

(52) U.S. Cl. CPC A61M 25/1029; A61F 2/958; B29C 65/32; B29C 66/91221; B29L 2031/7543; B29C 33/3842; A61F 2002/9583; A61F 2240/001; A61M 2025/1031; B29C 66/91411

(57) ABSTRACT

A method fixes an implant on a balloon. The balloon, together with an implant, which is crimped onto a balloon surface of the balloon such that the implant by an inner side contacts a contact region of the balloon surface, is provided in the interior of a mold. The balloon interior is acted on by a pressure and the implant is inductively heated so that the contact region over the implant is heated and plastically deformed. The inner side of the implant is embedded in the balloon surface. Additionally, an assembly is produced by the method and an apparatus are provided for carrying out the method.

### CROSS-REFERENCE TO RELATED APPLICATION

This application claims the priority; under 35 U.S.C. § 119, of German application DE 10 2018 120 940.1, filed Aug. 28, 2018; the prior application is herewith incorporated by reference in its entirety.

### BACKGROUND OF THE INVENTION

### Field of the Invention

The invention relates to a method and an apparatus for fixing an implant, preferably a stent, on a balloon of a balloon catheter, and to an assembly comprising an implant fixed on a balloon, and also an apparatus for carrying out the method. The present invention will be described on the basis of the example of an implant and a balloon of a balloon catheter. The present invention, however, is in principle suitable for fixing any implant on a balloon. Implants of this kind are for example heart valve prostheses with an implant-like basic structure, occluders, or generally tubular balloon-expandable implants.

In the case of implants crimped on balloons of a balloon catheter it is highly important that each implant is fixed securely to an assigned balloon of a balloon catheter so that the implant does not shift relative to the balloon during the implantation process. It is therefore important to attain a sufficient implant-holding force. This implant-holding force, in the case of an implant crimped on a balloon, is provided by the sum of interlocking and frictionally engaged connections.

In the relevant standards (for example ASTM standard F2394-07)—i.e. also within the scope of the present invention—a crimping of an implant/stent onto a balloon is understood to mean a securing of the implant on the balloon, wherein the implant is compressed on the balloon in the radial direction (towards the folded balloon) and in so doing is plastically deformed.

With regard to the implant-holding force, interlocking connections are created as a result of the engagement of at least two connection partners (here implant and balloon) in one another. The connection partners thus cannot detach from one another, even without force transfer or with an interrupted force transfer. In other words, in the case of an interlocking connection, one connection partner is in the way of the other connection partner.

Frictionally engaged connections presuppose a normal force on the surfaces to be connected to one another. Their mutual displacement is prevented, provided the counterforce brought about by the static friction is not exceeded. The force fit or friction fit is lost and the surfaces slip over one another when the tangentially acting loading force is greater than the static frictional force.

In U.S. Pat. No. 9,566,371 it is proposed to heat the implant and to place the balloon under pressure in order to weld the balloon to the implant. International patent disclosure WO 2017/011200 describes a heating and compression of a medical implant.

### SUMMARY OF THE INVENTION

On this basis, the object of the present invention is to provide a precise fixing of an implant, which is crimped onto a balloon, relative to the balloon in order in particular to prevent the risk of shifting of the implant, in particular during implantation. In particular, a method for more precise and quicker temperature control during the fixing process is sought.

This object is achieved by a method having the features of the independent method claim, an assembly having the features of the independent assembly claim, and by an apparatus having the features of the independent apparatus claim.

Embodiments of these aspects of the invention are specified in the corresponding independent claims and/or will be described hereinafter.

Accordingly, a method for fixing an implant on a balloon (for a balloon catheter) is disclosed. The balloon, which has an interior which can be acted on by pressure, together with an implant, which is crimped onto a balloon surface of the balloon such that the implant by use of an inner side contacts a contact region of the balloon surface, is provided in the interior of a mold. The balloon interior is acted on by a pressure and the implant is inductively heated so that the contact region between the balloon and the implant is heated and plastically deformed. The inner side of the implant is embedded in the balloon surface.

The contact region does not necessarily have to form a continuous area, but also can be composed of a plurality of separate contact points. Within the scope of the application the part of the balloon that is in direct contact with the structure of the implant is understood to constitute the contact region.

The method according to the invention is particularly suitable for fixing a stent on a balloon of a balloon catheter. Within the scope of this application a stent is understood to mean a permanent or degradable tubular structure which can be implanted in a bodily vessel, in particular a blood vessel. The stent may also have one or more coatings, in particular containing an active ingredient. Stents of this kind advantageously have multiple struts, which form the structure of the stent. The struts usually run in a meandering form and are arranged as a multiplicity of rings and/or helices and are connected to one another in such a way that they form a substantially cylindrical structure with a multiplicity of gaps or through-openings between the bars.

For inductive heating of the implant it is preferably provided that the implant is metallic or comprises at least one metallic component.

The implant (or preferably the stent) has in particular a multiplicity of through-openings, which each extend from the inner side of the implant to an outer side of the implant. Each through-opening has a circumferential lateral wall, via which the inner side of the implant is connected to the outer side of the implant, so that, by embedding the inner side of the implant in the balloon surface, regions of the balloon or the balloon surface associated with the through-openings protrude into the through-openings, thus producing an interlocking fit between the balloon and the implant. The balloon surface in particular also nestles closely at least in some sections against the wall or delimitation of the through-openings.

The through-openings of the implant are also referred to as cells of the implant. Each cell or through-opening is delimited by the implant, for example by interconnected struts of the implant. The struts can be connected to one another in one piece or integrally. In particular, the implant in this way can form a lattice structure. Such an implant can be formed for example by appropriate processing of a tubular (preferably metallic) preform, wherein the cells or through-openings are cut into the implant during the processing, for example by means of laser, so that struts with the cells (through-openings) are produced. However, it is also conceivable to produce the cells/through-openings of the implant in another way.

The plastic deformation of the heated contact region is achieved in particular in that a pressure is applied in the balloon interior and an inner side of the mold surrounding the implant forms an abutment for the implant, i.e. the outer side of the implant can contact or press against the inner side of the mold.

In accordance with an embodiment of the method according to the invention it is provided that the implant is heated inductively by an inductor. The inductor can be, for example, a helical inductor, a fork inductor or a folding inductor. Other inductor designs are also conceivable. In accordance with an embodiment of the invention the inductor is preferably furthermore formed so as to be compatible with clean rooms.

The inductor for example can have power consumption in the region of 2 kW and an operating frequency that lies for example in the range of from 70 kHz to 450 kHz.

In accordance with an embodiment of the method according to the invention it is furthermore provided that the mold contains an electrically non-conductive material. In particular, the mold can comprise one of the following materials or can consist of one of the following materials: glass, a ceramic, or a heat-resistant plastic, such as PEEK (polyether ether ketone), Teflon, etc.

Furthermore, in accordance with an embodiment of the method according to the invention it is provided that the implant or the contact region is heated to a target temperature during the inductive heating.

It is furthermore provided in accordance with an embodiment of the method according to the invention that an actual temperature of the implant during heating of the implant to the target temperature is measured continuously or repeatedly, and the actual temperature of the implant (during heating of the implant) is controlled until it reaches the target temperature.

It is furthermore provided in accordance with an embodiment of the method according to the invention that the actual temperature of the implant during heating of the implant to the target temperature is measured continuously or repeatedly with the aid of a measurement of heat radiation emitted by the implant. The temperature measurement is therefore performed in other words contactlessly. In particular, the heat radiation or infrared radiation is measured using a pyrometer in order to determine the temperature of the implant.

In accordance with an embodiment of the method according to the invention it is furthermore provided that the mould has a through-opening for transmission of the heat radiation to be measured.

The through-opening can have a diameter in the range of from, for example, 0.3 mm to 0.5 mm.

It is furthermore provided in accordance with an embodiment of the method according to the invention that the pressure in the balloon interior lies in a range of from 10 bar to 30 bar, and in particular is 15 bar.

It is furthermore provided in accordance with an embodiment of the method according to the invention that the target temperature lies in one of the following ranges below the melting point:

in the range of from 40° C. to 150° C.,

in the range of from 40° C. to 140° C. (in particular if the balloon is made of PA 12 (see below)),

in the range of from 60° C. to 150° C. (in particular if the balloon is made of Pebax 7033 (see below)),

in the range of 50° C. to 110° C.,

in the range of from 100° C. 110° C., in particular 102° to 107° C. (in particular if the material of the balloon is Pebax 7300 and/or in the case of a metallic implant or stent to which in particular no medicament has been applied), and

in the range of from 50° C. to 60° C., in particular 53° C. to 57° C. (in particular if the material of the balloon is PA 12 and/or a medicament has been applied to the implant or stent, i.e. the stent is in particular what is known as a drug eluting stent (DES), i.e. a stent that is configured to release a medicament, in contrast to an uncoated stent (BMS)).

It is furthermore provided in accordance with an embodiment of the method according to the invention that the balloon is manufactured from a balloon material or comprises a balloon material, in particular at the balloon surface, which balloon material has a glass transition temperature. The target temperature is greater than or equal to the glass transition temperature, and wherein in particular the target temperature deviates by no more than 10%, in particular by no more than 5%, in particular by no more than 1% from the glass transition temperature. The glass transition temperature can be determined by a method known to a person skilled in the art, for example thermoanalytical methods (DSC, DMA, DIL, LFA).

In accordance with an embodiment of the present invention the balloon is formed of a material such as polyamide or modifications thereof, for example a polyether block amide (for example PEBAX®). The material may furthermore be a thermoplastic elastomer, for example TPE-A. These partly crystalline plastics (many conventional plastics have a crystalline fraction of from 10% to 80%) have both a glass transition temperature below which the amorphous phase freezes (associated with embrittlement) and a melting point at which the crystalline phase dissolves. The melting point separates the entropic elasticity range clearly from the flow range.

In accordance with an embodiment of the present invention the balloon can be manufactured from, or can consist of, in particular one of the following materials: a polyamide, in particular PA 12, for example Grilamid polyamide 12 L25; a polyether block amide (Peba), for example PEBAX® 3533 or PEBAX® 7033; PET; PEEK; TPU.

Grilamid polyamide 12 L25 is in particular a partly crystalline thermoplastic with a glass transition temperature of 37° C. and with a melting point of 178° C. Peba, or rather PEBAX® 3533 (CAS No. 77402-38-1) or PEBAX® 7033 (CAS No. 77402-38-1), is a partly crystalline thermoplastic elastomer (TPE), wherein for example PEBAX® 3533 has a glass transition temperature of −65° C. and a melting point of 144° C. Furthermore, PET (polyethylene terephthalate) can have, for example, a glass transition temperature of 70° C. and a melting point of for example 255° C. PEEK (polyether ether ketone) is in particular likewise a partly crystalline thermoplastic with a glass transition temperature of, for example, 143° C. and a melting point of for example 340° C. Furthermore, thermoplastic polyurethane (TPU) can be used as a material for the balloon within the scope of the present invention.

Furthermore, it is provided in accordance with an embodiment of the method according to the invention that the implant or the stent, when the balloon interior is acted on by the pressure, is exposed to the target temperature over a period of time of at least 10 s, in particular at least 20 s, in particular at least 30 s, in particular at least 40 s, in particular at least 50 s, in particular at least 60 s.

In accordance with an embodiment of the method it is provided that the balloon, when the balloon interior is acted on by the pressure and under the negative pressure applied in the interior of the sleeve, is exposed to said target temperature over a period of time of at least 10 s to 100 s, in particular 20 s to 80 s, in particular 30 s to 60 s, in particular 20 s to 40 s, in particular 25 s to 35 s, in particular 50 s to 70 s, in particular 55 s to 65 s.

The periods of time 20 s to 40 s or 25 s to 35 s are used in particular when the implant is a stent that is configured to release a medicament (DES). In this case the balloon can consist for example of PA 12 or another polyamide.

The periods of time 50 s to 70 s or 55 s to 65 s are used in particular when the implant is a fully metal stent or implant (BMS). In this case the balloon can consist for example of PEBAX® 7033 or another TPE, in particular TPE-A.

With regard to the different stent configurations (DES or BMS) or corresponding implants, the present invention can be implemented in particular with the following exemplary parameters:

| | | End | Period of time | Pressure |
|---|---|---|---|---|
| Stent/ | Balloon | temperature | or process | in the balloon |
| Implant | material | ° (C.) | duration (s) | interior (bar) |
| BMS | Pebax 7033 | 105 +/− 3 | 60 | 15 +/− 0.5 |
| DES | PA 12 | 55 +/− 2 | 30 | 15 +/− 0.5 |

It is furthermore provided in accordance with an embodiment of the method according to the invention that the balloon interior is acted on by the pressure before the implant or the contact region is heated. The application of pressure, however, could also be performed or initiated during heating of the implant or once the implant has been heated to the target temperature.

The present invention also relates to an assembly comprising an implant that is crimped onto a balloon surface of a balloon (in particular a balloon of a balloon catheter), wherein the implant is fixed to the balloon by the method according to any one of the preceding claims.

Furthermore, one aspect of the present invention relates to an apparatus for fixing an implant to a balloon. This apparatus is suitable in particular for carrying out the method according to the invention and is preferably used in the method according to the invention.

The apparatus according to the invention comprises at least:

a mold, which has an interior for receiving an implant crimped onto a balloon,

a device which can be brought into fluidic connection to the interior of the balloon and which is designed to act on the interior of the balloon with a pressure (for example by introducing compressed air), and

an inductor, which is configured to heat the implant inductively or contactlessly when the balloon and the implant crimped thereon are arranged in the interior of the mould.

The mold for example can be cylindrical at least in some sections and can have a circumferential wall which is designed to surround the implant when this is arranged together with the balloon in the interior of the mold. The wall delimits the interior and has an inner side which faces the interior of the mold. The inner side is used in particular as a contact face or abutment for the implant. The balloon material or the balloon surface can thus press again the implant or the inner side thereof by applying a pressure in the balloon interior, wherein the implant is held back in the radial direction by the inner side of the mould.

In accordance with an embodiment of the apparatus according to the invention it is provided that the apparatus has a temperature sensor for measuring a momentary temperature (i.e. actual temperature) of the implant. The temperature sensor in particular is designed to measure, control and keep constant the temperature with the aid of a measurement of a heat radiation emitted by the implant. The temperature sensor can be configured for example as a pyrometer.

It is furthermore provided in accordance with the apparatus according to the invention that the mould has a through-opening which allows the heat radiation from the implant to the temperature sensor to pass through. The through-opening in particular is arranged between the implant and the temperature sensor, more specifically in particular in the circumferential wall of the mold.

Other features which are considered as characteristic for the invention are set forth in the appended claims.

Although the invention is illustrated and described herein as embodied in a method and an apparatus for embedding an implant in a balloon surface by inductive heating of the implant, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of an apparatus which can be used to carry out the method according to the invention;

FIG. 2 is a schematic sectional depiction of a stent to be fixed to a balloon; and

FIG. 3 is a perspective view of an inductor of the apparatus for carrying out the method according to the invention, wherein the inductor surrounds the mold of the apparatus helically.

### DETAILED DESCRIPTION OF THE INVENTION

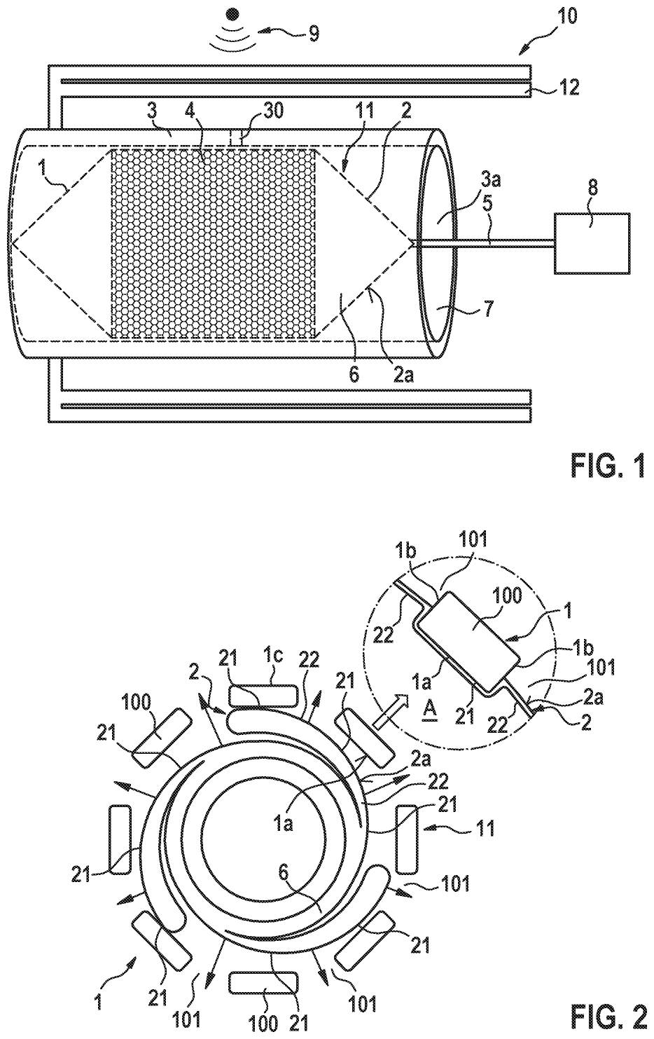

Referring now to the figures of the drawings in detail and first, particularly to FIGS. 1 and 2 thereof, there is shown an embodiment of an apparatus 10 which is configured to carry out a method according to the invention. The apparatus 10 therefore has, for example, a cylindrical mold 3 with an inner side 3a, which faces an interior 7 of the mold 3. The inner side 3a forms a contact face for a stent 1 which can be arranged in the interior 7 and which is crimped onto a balloon surface 2a of an in particular folded balloon 2 and which in addition is to be embedded in the balloon surface 2a by use of the apparatus 10 so as to produce an interlocking fit increasing the stent-holding force. The apparatus 10 furthermore has in particular a device 8 which can be brought into fluidic connection to an interior 6 of the balloon 2 and is configured to act on the interior 6 of the balloon 2 with a pressure. The device can be configured for example as a compressed gas source, in particular a compressed air source 8, which is configured to introduce a gas, in particular compressed air, in the balloon interior 6 in order to generate the pressure in the balloon interior 6.

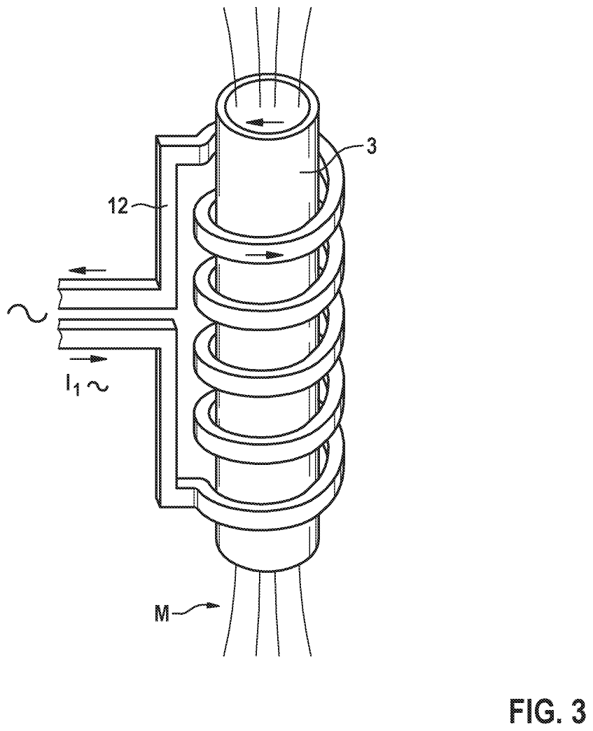

The apparatus 10, as shown by way of example in FIG. 3, furthermore has at least one inductor 12, which is configured to heat the stent 1 directly, more specifically contactlessly, when the balloon 2 and the stent 1 crimped thereon are arranged in the interior 7 of the mold 3. The inductor 12 is configured to generate a magnetic field M, which generates in the stent 1 an electric current, which in turn generates Joule heat, which heats the stent 1. In accordance with FIG. 3, the inductor 12 is helical, wherein the inductor 12 extends helically around the mold 3, such that the stent 1 arranged therein is heatable by means of the inductor 12. Instead of a helical inductor 12, other inductors known to a person skilled in the art can also be used, for example a fork inductor or a folding inductor. In the case of a fork inductor the conductor of the inductor is arranged such that the inductor has an open side, on which the object to be heated can be arranged in relation to the inductor such that the inductor extends on two sides of the object facing away from one another. A folding inductor allows the inductor to be opened via a hinge, such that the object can be placed in the inductor in the open state of the folding inductor and is surrounded by the inductor when it is closed.

By directly heating the stent 1, this heats the balloon surface 2a or a corresponding balloon material merely in a contact region 21 between an inner side 1a of the stent 1 and the balloon surface 2a,

The stent 1 or the contact region 21 is heated here such that it is plastically deformable. The stent 1 is pressed or embedded via its inner side 1a into the balloon surface by way of the applied pressure in the balloon interior

Outside the contact region 21 the balloon is not heated or is heated to a considerably smaller extent, and therefore the material outside the contact region can remain at least in part in a plastically non-deformable state,

As can also be seen with reference to FIG. 2, the stent 1 can have multiple through-openings 101, which each extend from the inner side 1a of the stent 1 to an outer side 1c of the stent 1. Each through-opening 101 has a circumferential lateral wall or delimitation 1b formed by the stent 1, via which wall or delimitation the inner side 1a of the stent 1 is connected to the outer side 1c of the stent 1, so that, by embedding the inner side 1a of the stent 1 in the balloon surface 2a, regions 22 of the balloon 2 or the balloon surface 2a associated with the through-openings 101 protrude into the through-openings 101 (this is indicated by arrows in the schematic depiction according to FIG. 2), wherein an interlocking fit is produced between the balloon 2 and the stent 1, The balloon surface 2a in particular also nestles closely at least in some sections against the lateral wall or delimitation 1b of the through-openings 101, as is shown in particular in the detail A of FIG. 2. The through-openings 101 can each be delimited in particular by struts 100 of the stent 1, which then also form the lateral walls or delimitations 1b.

In order to be able to adjust or control the temperature of the stent 1 in the method according to the invention, it is provided in particular to measure the temperature by means of a temperature sensor 9. The temperature sensor in particular is a temperature sensor which, in order to determine the temperature of the stent 1, is configured to measure heat radiation (for example infrared radiation) emitted by the stent 1. To this end, a wall of the mold 3 can have a through-opening 30, which allows heat radiation of the stent 1 to escape unhindered, in such a way that it can be detected by the temperature sensor 9 and can be evaluated in order to determine the actual temperature of the stent 1. The temperature sensor 9 is preferably connected to the inductor 12 via a control unit (not shown) in order to control the temperature of the stent 1.

By way of the solution according to the invention an optimal interlocking connection between the stent 1 and balloon 2 is thus produced, which increases the stent-holding force, wherein in particular the balloon material is warm and flexible only in the contact region 21 with the stent 1. The stent 1 hereby can be embedded deep in the balloon material. The risk of the stent shifting (see “Stent Displacement” according to ASTM F2394-07) and the risk of the stent detaching from the balloon (see “Stent Dislodgment” according to ASTM F2394-07) thus reduces, advantageously.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A method for fixing an implant on a balloon, which comprises the steps of:
providing the balloon, together with the implant which is crimped onto a balloon surface of the balloon such that the implant by means of an inner side contacts a contact region of the balloon surface, in an interior of a mold;
applying pressure to a balloon interior; and
inductively heating the implant so that the contact region between the balloon and the implant is heated and plastically deformed, wherein the inner side of the implant is embedded in the balloon surface.

2. The method according to claim 1, which further comprises inductively heating the implant by means of an inductor.

3. The method according to claim 1, wherein the mold is formed from an electrically non-conductive material.

4. The method according to claim 1, which further comprises forming the mold from at least one material selected from the group consisting of glass, a ceramic, and a heat-resistant plastic.

5. The method according to claim 1, which further comprises heating the implant to a target temperature during the inductive heating.

6. The method according to claim 5, which further comprises:
measuring a current temperature of the implant during the inductive heating of the implant; and
controlling an actual temperature of the implant during the inductive heating of the implant until it reaches the target temperature.

7. The method according to claim 6, which further comprises measuring and controlling the actual temperature of the implant during the inductive heating of the implant with an aid of a measurement of heat radiation emitted by the implant.

8. The method according to claim 7, wherein the mold has a through-opening for a transmission of the heat radiation to be measured.

9. The method according to claim 1, wherein the pressure in the balloon interior lies in a range of from 10 bar to 30 bar.

10. The method according to claim 5, which further comprises selecting the target temperature to be in a range of:
40° C. to 150° C.;
40° C. to 140° C.;
60° C. to 150° C.;
50° C. to 110° C.;
100° C. to 110° C.;
102° C. to 107° C.;
50° C. to 60° C.; or
53° C. to 57° C.

11. The method according to claim 5, which further comprises manufacturing the balloon from a balloon material which has a glass transition temperature, wherein the target temperature is greater than or equal to the glass transition temperature, and wherein the target temperature deviates by no more than 10% from the glass transition temperature.

12. The method according to claim 5, which further comprises manufacturing the balloon from a balloon material which has a glass transition temperature, wherein the target temperature is greater than or equal to the glass transition temperature, and wherein the target temperature deviates by no more than 5% from the glass transition temperature.

13. The method according to claim 5, which further comprises manufacturing the balloon from a balloon material which has a glass transition temperature, wherein the target temperature is greater than or equal to the glass transition temperature, and wherein the target temperature deviates by no more than 1% from the glass transition temperature.

14. The method according to claim 5, wherein the implant, with the balloon interior acted on by the pressure, is exposed to the target temperature being constant over a period of time of at least 10 s to 100 s.

15. The method according to claim 5, wherein the implant, with the balloon interior acted on by the pressure, is exposed to the target temperature being constant over a period of time of at least 20 s to 80 s.

16. The method according to claim 5, wherein the implant, with the balloon interior acted on by the pressure, is exposed to the target temperature being constant over a period of time of at least 30 s to 60 s.

17. The method according to claim 5, wherein the implant, with the balloon interior acted on by the pressure, is exposed to the target temperature being constant over a period of time of at least 25 s to 35 s.

18. The method according to claim 5, wherein the implant, with the balloon interior acted on by the pressure, is exposed to the target temperature being constant over a period of time of at least 55 s to 65 s.

19. An assembly, comprising:
a balloon having a balloon surface; and
an implant crimped onto said balloon surface of said balloon, wherein the implant is fixed to said balloon by means of the method according to claim 1.

20. An apparatus for fixing an implant to a balloon, the apparatus comprising:
a mold having an interior for receiving the implant crimped onto the balloon;
a device which can be brought into fluidic connection to a balloon interior of the balloon and configured to act on the balloon interior of the balloon with pressure; and
an inductor configured to heat the implant when the balloon and the implant crimped thereon are disposed in an interior of said mold.
